(19) United States
(12) Patent Application Publication
(10) Pub. No.: US 2013/0158391 A1
(43) Pub. Date: Jun. 20, 2013

(54) IMAGE PROCESSING UNIT FOR OPTICAL TOMOGRAPHY

(71) Applicants: Wai-Chi Fang, Hsinchu City (TW); Tien-Ho Chen, Hsinchu City (TW); Shih Kang, Hsinchu City (TW); Shih-Yang Wu, Hsinchu City (TW); Ching-Ju Cheng, Hsinchu City (TW)

(72) Inventors: Wai-Chi Fang, Hsinchu City (TW); Tien-Ho Chen, Hsinchu City (TW); Shih Kang, Hsinchu City (TW); Shih-Yang Wu, Hsinchu City (TW); Ching-Ju Cheng, Hsinchu City (TW)

(73) Assignee: NATIONAL CHIAO TUNG UNIVERSITY, Hsinchu City (TW)

(21) Appl. No.: 13/524,385

(22) Filed: Jun. 15, 2012

(30) Foreign Application Priority Data
Dec. 14, 2011 (TW) 100146130

(51) Int. Cl. A61B 6/02

(52) U.S. Cl. 600/425

(57) ABSTRACT

An image processing unit for optical tomography is applicable in miniaturized diffusion optical tomography devices. It includes an image reconstructor and an image post-processor. The image reconstructor receives a plurality of optical signals generated from an reaction of an object under test with irradiating light and an inverse solution matrix of an image model of the object, and obtains scalar product on each of the optical signals and the inverse solution matrix through a sub-frame algorithm to generate an original image corresponding to the object. Then, the image post-processor performs a Gaussian extended algorithm on the original image to output a final image. With the image reconstruction and image post-processing of the present invention, slowing down of computation speed due to miniaturization of the conventional optical tomography techniques can be avoided, while providing good quality in the output image.

### FIELD OF THE INVENTION

The present invention relates to an image processing unit for optical tomography, and more particularly, to an image processing unit applicable to portable diffusion optical tomography devices.

### BACKGROUND OF THE INVENTION

In various current techniques for diagnosing chest or brain tumors, diffusion optical tomography has become a popular method for its non-intrusiveness and real-time imaging.

In particular, diffusion optical tomography utilizes the fact that body tissues or tumors exhibit different optical properties (e.g. absorption, reflection and deflection) to excitation light with specific wavelengths and thus the differences in tissues and inner structure of the human body can be identified. For example, oxygenated and non-oxygenated hemoglobin have different levels of absorption to near-infrared light. Thus, such characteristics can be used in clinic trials related to blood flow, blood volume and oxygen saturation concentration, and also for determination of body tissues or tumors as just mentioned. Therefore, the use of near-infrared light in diffusion optical tomography creates more benefits and extends the application range of the diffusion optical tomography.

In recent years, along with research developments and advances in manufacturing technologies, attentions have been focused on improving image reconstruction techniques after optical tomography. In other words, in order to meet the requirement for high image resolution, extremely large computations often have to be performed on the tomography results. However, huge computation results in long imaging time. As a result, more components are added to speed up the calculations, rendering an oversized device that cannot be easily moved around, compromising its mobility.

It is understood from the above that the abovementioned shortcoming of the diffusion optical tomography can be improved by reducing the diffusion optical tomography device. However, reducing the size of the device lengthens the imaging time, thus modifications of software, hardware or firmware are also needed to realize a reliable and efficient image reconstruction. Thus, there is a need to provide a good image reconstruction technique in miniaturized diffusion optical tomography devices or equipment.

### SUMMARY OF THE INVENTION

In light of the foregoing drawbacks, an objective of the present invention is to an image processing unit for optical tomography that can be applied to a miniaturized diffusion optical tomography device, providing good image reconstruction effect under reduced size.

In accordance with the above and other objectives, the present invention provides an image processing unit for optical tomography, which includes: an image reconstructor for receiving a plurality of optical signals generated from an reaction of an object with irradiating light and an inverse solution matrix of an image model of the object, and performing correlation calculations on each of the optical signals and the inverse solution matrix to generate an original image corresponding to the object; and an image post-processor for performing a Gaussian extended algorithm on the original image to output a post-processed final image.

In an embodiment, the aforementioned image reconstructor further includes: an optical signal buffer, an object information buffer and an image reconstruction module. The optical signal buffer is used for buffering the plurality of optical signals. The object information buffer is used for buffering the inverse solution matrix. The image reconstruction module is used for processing each optical signal through a sub-frame algorithm to obtain detection data of the object under test, and obtaining a scalar product of the detection data and the inverse solution matrix to reconstruct the original image.

In another embodiment, the aforementioned image post-processor further includes: an input buffer and an image processing module. The input buffer is used for buffering the original image. Then, the image processing module performs image smoothing process on the original image based on weighted arrays formed by the Gaussian extended algorithm to generate the final image.

In addition, the image model of the object is established by optical parameters of the object. The inverse solution matrix can be obtained by performing singular value decomposition on the image model.

In yet another embodiment, the image reconstructor and the image post-processor in the image processing unit for optical tomography are realized by hardware circuits.

Compared to the prior art, the image processing unit for optical tomography according to the present invention can be used in miniaturized diffusion optical tomography devices. More particularly, the image reconstruction involves combines and calculates each optical signal retrieved and the image model of the object, and post-processes the original image generated to increase the image resolution and improve the image continuity. The image processing unit for optical tomography can be realized by a chip, which enables the diffusion optical tomography devices to become portable, low-cost and efficient equipment suitable for home health care.

### BRIEF DESCRIPTION OF THE DRAWINGS

The present invention can be more fully understood by reading the following detailed description of the preferred embodiments, with reference made to the accompanying drawings, wherein:

FIG. 1 is a block diagram illustrating an image processing unit for optical tomography according to an embodiment of the present invention;

FIG. 2 is a block diagram illustrating details of an image reconstructor in an image processing unit for optical tomography according to an embodiment of the present invention;

FIG. 3 is a block diagram illustrating details of an image post-processor in an image processing unit for optical tomography according to an embodiment of the present invention; and

FIG. 4 is a block diagram illustrating an image processing unit for optical tomography applied to a diffusion optical tomography device according to an embodiment of the present invention.

### DETAILED DESCRIPTION OF THE EMBODIMENTS

The present invention is described by the following specific embodiments. Those with ordinary skills in the arts can readily understand the other advantages and functions of the present invention after reading the disclosure of this specification. The present invention can also be implemented with different embodiments. Various details described in this specification can be modified based on different viewpoints and applications without departing from the scope of the present invention.

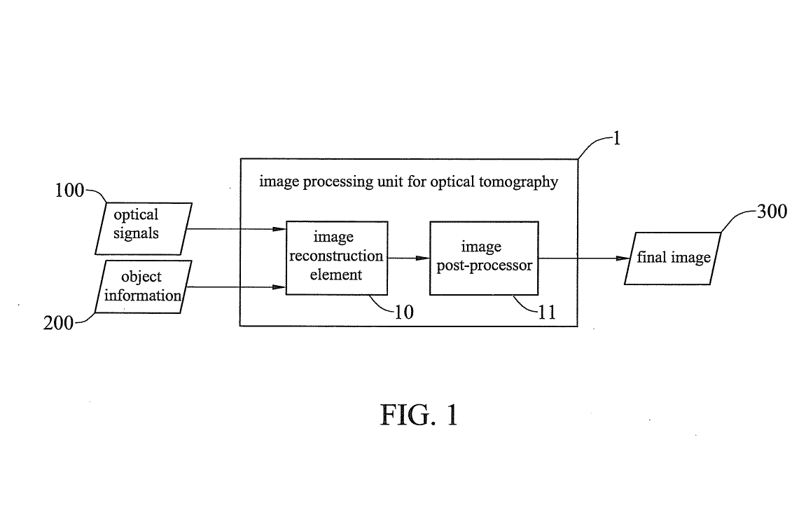

Referring to FIG. 1, a block diagram illustrating an image processing unit for optical tomography according to an embodiment of the present invention is shown. As shown, the image processing unit for optical tomography 1 is applicable within a diffusion optical tomography device. The image processing unit for optical tomography 1 mainly includes an image reconstruction element 10 and an image post-processor 11.

It should be noted that in order to facilitate use in home health care, the present invention proposes an image processing unit for a small but high-performance miniaturized diffusion optical tomography device. With the reduction in the size of the device, traditional approach of using software to perform immense calculations required for image processing is no longer appropriate. Thus, the image reconstruction element 10 and the image post-processor 11 of the present invention can be realized by hardware circuits, such as those embedded in a chip, thereby meeting the needs for both miniaturization and high performance. However, this is only a preferred embodiment, and the image processing unit for optical tomography 1 of the present invention is not limited to this.

The image reconstruction element 10 is used for receiving a plurality of optical signals 100 from an reaction of an object under test with irradiating light and object information 200 inputted in advance. In this embodiment, the object information 200 is an inverse solution matrix generated by an image model of the object, and the image reconstruction element 10 performs correlation calculations on each of the optical signals 100 and the inverse solution matrix, for example, a sub-frame algorithm is used to compute scalar product of the optical signals 100 and the inverse solution matrix, which generate a corresponding original image of the object.

In implementations, the object information 200 is an inverse solution matrix calculated from an image model of the object. The image model of the object can be established by optical parameters of the object that are data inputted in advance. These optical parameters may include measurement depth, adsorption coefficient, reflection coefficient, diffusion coefficient, etc. Then, singular value decomposition is computed on the image model to obtain the inverse solution matrix. The method for obtaining the inverse solution matrix is not the focus of the present invention, and thus will not be further described.

In addition, the diffusion optical tomography device emits a plurality of light sources such as near-infrared light to the object. The light reacts with the object and is reflected and received by a detector. In other words, the plurality of optical signals 100 generated from an reaction of an object under test with the irradiating light indicate biological signals in different regions of the object. For example, the object is the inner structure of a human body, after the near-infrared light is irradiated on the human body, different structures in the body may absorb and reflect different levels of the near-infrared light. The detector then detects the optical signals 100 returned resulting from light emitted by each light source, thereby detecting the differences in the inner structure of the human body.

Thus, all light sources are regionally divided, so that the optical signal 100 for the region onto which the light irradiates can be individually calculated. This is different from the prior art, which calculates sensing information from all light sources in a single calculation and results in very long computation time. The image reconstruction element 10 obtains the scalar product of the received optical signals 100 and the inverse solution matrix of the preset image model of the object to obtain the complete original image of the object.

The image post-processor 11 performs a Gaussian extended algorithm on the original image generated by the image reconstruction element 10 to output a processed final image 300. Specifically, since the image reconstruction element 10 regionally divides the light sources, the separate calculation for each individual light source results in discontinuities at the boundary of images for two neighboring regions in the overall image, so post processing is carried out on the original image generated by the image reconstruction element 10, such as a smooth process or increasing of image resolution is performed at the boundary of two image regions, so the final image 300 will have a better representation.

It can be understood from the above that, in order to overcome the very long computation time resulted from using software to perform extremely large computations and oversized equipment, the present invention designs the image reconstruction element 10 and the image post-processor 11 as hardware chips so as to perform calculations on the optical signals and the image model to generate the original image and especially speeds up the calculations by separating calculations for each light source, and optimizes the final image through post-image processing, thereby enabling a miniaturized and efficient diffusion optical tomography device.

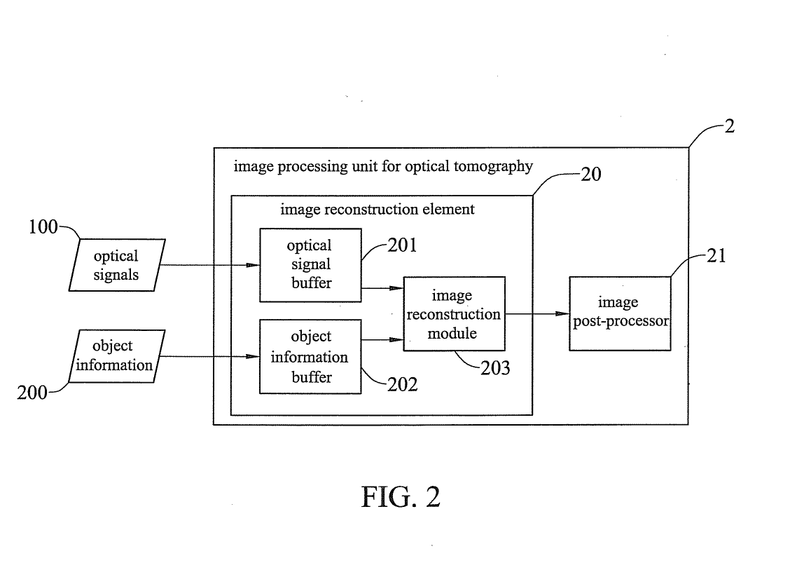

FIG. 2 is a block diagram illustrating details of an image reconstruction element in an image processing unit for optical tomography according to an embodiment of the present invention. In FIG. 2, an image processing unit for optical tomography 2 provides functions such as image reconstruction and image post-processing, wherein an image reconstruction element 20, an image post-processor 21, optical signals 100 and object information 200 are the same as those described with respect to FIG. 1. The image reconstruction element 20 further includes an optical signal buffer 201, an object information buffer 202 and an image reconstruction module 203. However, the aforementioned module or structure is not to be constructed in a limiting sense, and can be adjusted according to needs.

The optical signal buffer 201 is used for buffering the plurality of optical signals 100, and the object information buffer 202 is used for buffering the object information 200, that is, the aforementioned inverse solution matrix. The optical signal buffer 201 and the object information buffer 202 primarily provide buffering of the optical signals 100 and the object information 200. This avoids computation problems resulting from two entries of information entering at different times, and can be used for repetitive readings.

The image reconstruction module 203 processes each optical signal 100 through a sub-frame algorithm to obtain detection data of the object, and obtains a scalar product of the detection data and the inverse solution matrix to reconstruct the original image. The image reconstruction module 203 is the operation core of the image reconstruction element 20. The image reconstruction module 203 converts each optical signal 100 into digital detection data, obtains the scalar product of the detection data and the inverse solution matrix to get the original image of the object and transmits this original image to the image post-processor 21. In addition, the image reconstruction element 20 further includes a control module (not shown) connected with the image reconstruction module 203 and the optical signal buffer 201 to provide control of the analog-to-digital conversion of the optical signals 100 and the image reconstruction process.

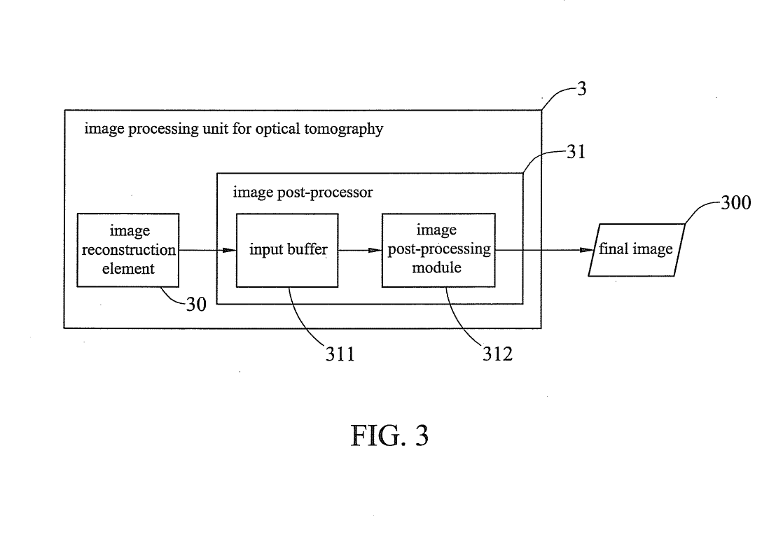

FIG. 3 is a block diagram illustrating details of an image post-processor in an image processing unit for optical tomography according to an embodiment of the present invention. In FIG. 3, an image processing unit for optical tomography 3 also provides functions such as image reconstruction and image post-processing, wherein an image reconstruction element 30, an image post-processor 31, optical signals 100 and object information 200 are the same as those described with respect to FIG. 1. The image reconstruction element 30 further includes an input buffer 311 and an image processing module 312. However, the aforementioned module or structure is not to be constructed in a limiting sense, and can be adjusted according to needs.

The input buffer 311 is used for buffering the original image generated by the image reconstruction element 30. This provides a similar function with the optical signal buffer 201 and the object information buffer 202 described in FIG. 2.

The image processing module 312 performs an image smoothing process on the original image based on weighted array formed by the Gaussian extended algorithm to obtain the final image 300. More specifically, the image processing module 312 is the operating core of the image post-processor 31. The image processing module 312 performs image post-processing on the original image to obtain an image that can be easily viewed by human eyes. The image smoothing process performed by the image processing module 312 enables boundaries of two neighboring optical signals to have image continuity. Alternatively, the image processing module 312 can increase image resolution so that the final image 300 will have a better appearance. The image post-processing may include numerous approaches such as fine tuning of photo parameters (e.g. saturation, contrast, sharpness etc.) or an image edge smoothing process to avoid any observable discontinuities at boundaries. In addition, the image post-processor 31 further includes a control module (not shown) connected with the image processing module 312 for providing control of the image post-processing.

The application of the image processing unit for optical tomography described with respect to FIGS. 1-3 in a diffusion optical tomography device is discussed as follows.

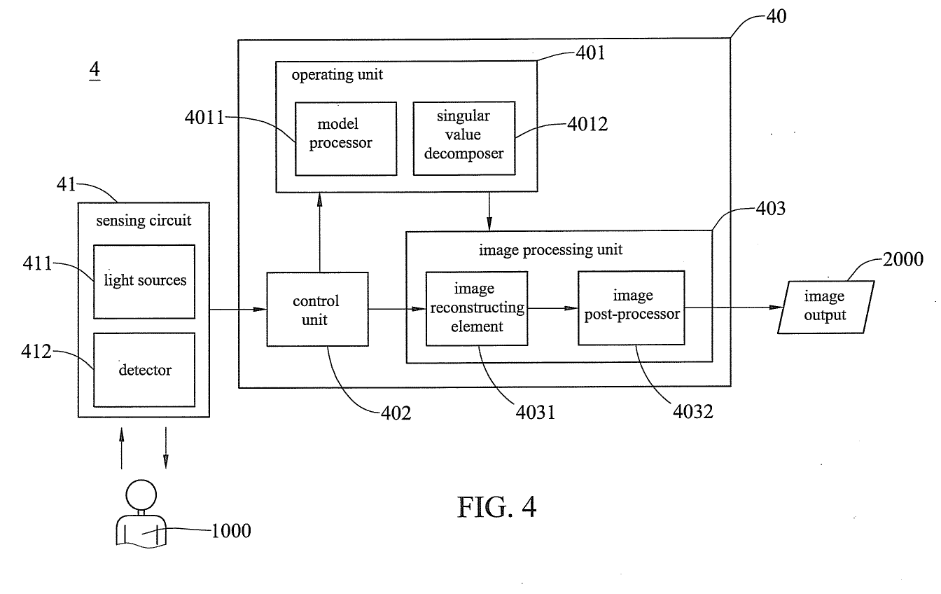

FIG. 4 is a block diagram illustrating an image processing unit for optical tomography applied to a diffusion optical tomography device according to an embodiment of the present invention. In FIG. 4, a diffusion optical tomography device 4 is a miniaturized device that is easy to carry and small in size. Compared to the large equipment employed for software computations in the prior art, the diffusion optical tomography device 4 is more advantageous.

The diffusion optical tomography device 4 includes an optical tomography element 40 and a sensing circuit 41. The sensing circuit 41 is electrically connected to light sources 411 and a detector 412. The light sources 411 emit light such as near-infrared light to a human chest 1000, and the reflected light is then detected by the detector 412 to generate the aforementioned optical signals. That is, the sensing circuit 41 obtains the optical signals corresponding to the inner structure of the human chest 1000, and transmits them to the optical tomography element 40.

The optical tomography element 40 primarily performs the image reconstruction and image post-processing. The optical tomography element 40 includes an operating unit 401, a control unit 402 and an image processing unit 403. The control unit 402 primarily controls the operations of various units within the optical tomography element 40. The operating unit 401 performs pre-processing on the optical information of the object so as to generate the original image of the object in combination with the sensed optical signals. The image processing unit 403 performs the image reconstruction and image post-processing.

More specifically, a user can input optical parameters related to an object under test via a user control interface (not shown). Meanwhile, a model processor 4011, based on the control by the control unit 402, establishes an image model of the object based on the optical parameters of the object, that is, converts the received optical parameters of the object into factors used for matrix calculation, and establishes a model matrix of the object based on the factors used for matrix calculation and some preset basic information in the sensing circuit 41. Then, a singular value decomposer 4012 performs singular value decomposition on the model matrix to obtain an inverse solution matrix of the object. The inverse solution matrix is combined with the sensed optical signals to generate the original image of the object.

The image processing unit 403 is the aforementioned image processing core technique of the present invention. After the inverse solution matrix generated by the operating unit 401 and the optical signals sensed by the sensing circuit 41 are received, an image reconstructing element 4031 performs image reconstruction, and then an image post-processor 4032 performs image post-processing on the original image of the object generated to obtain an image output 2000 with a better effect.

Therefore, the overall diffusion optical tomography device 4 can be a circuit design and manufactured as chips for miniaturization. Especially, fast and efficient image processing can be achieved through image reconstruction and image post-processing by the image reconstructing element 4031 and the image post-processor 4032 in the image processing unit 403.

In summary, the image processing unit for optical tomography of the present invention is primarily used for image reconstruction and image post-processing that combines and calculates each optical signal and the image model of the object, that is, obtains the scalar product using the sub-frame algorithm to obtain the original image of the object Then, the Gaussian extended algorithm is performed on the original image to increase the resolution of the image and achieve continuities between images in each zones, thus obtaining a better image output. The image processing unit for optical tomography of the present invention is realized as a chip, so that the size of the device can be minimized, and the chip is relatively cheap with fast processing speed. This makes it suitable for application in home or portable health care equipment, and is clearly more advantageous than the large optical tomography device employing software calculations currently used.

The above embodiments are only used to illustrate the principles of the present invention, and they should not be construed as to limit the present invention in any way. The above embodiments can be modified by those with ordinary skill in the art without departing from the scope of the present invention as defined in the following appended claims.

What is claimed is:

1. An image processing unit for optical tomography, comprising:
an image reconstructor for receiving a plurality of optical signals generated from an reaction of an object with irradiating light and an inverse solution matrix of an image model of the object, and performing correlation calculations on each of the optical signals and the inverse solution matrix to generate an original image corresponding to the object; and
an image post-processor for performing a Gaussian extended algorithm on the original image to output a final image.

2. The image processing unit for optical tomography of claim 1, wherein the plurality of optical signals generated from the reaction of the object with the irradiating light are biological signals of different regions in the object.

3. The image processing unit for optical tomography of claim 1, wherein the image reconstructor further comprises:
an optical signal buffer for buffering the plurality of optical signals;
an object information buffer for buffering the inverse solution matrix; and
an image reconstruction module for processing each optical signal through a sub-frame algorithm to obtain detection data of the object, and obtaining a scalar product of the detection data and the inverse solution matrix to reconstruct the original image.

4. The image processing unit for optical tomography of claim 1, wherein the image post-processor further comprises:
an input buffer for buffering the original image; and
an image processing module for performing image smoothing process on the original image based on weighted arrays formed by the Gaussian extended algorithm to generate the final image.

5. The image processing unit for optical tomography of claim 4, wherein the image smoothing process performed by the image processing module enables a boundary between two neighboring regions irradiated by optical signals to have an image continuity.

6. The image processing unit for optical tomography of claim 4, wherein the image processing module increases the image resolution.

7. The image processing unit for optical tomography of claim 1, wherein the inverse solution matrix is obtained by performing singular value decomposition on the image model.

8. The image processing unit for optical tomography of claim 1, wherein the image model is established by optical parameters of the object.

9. The image processing unit for optical tomography of claim 8, wherein the optical parameters of the object comprises one or more of measurement depth, adsorption coefficient, reflection coefficient and diffusion coefficient.

10. The image processing unit for optical tomography of claim 1, wherein the image reconstructor and the image post-processor are implemented by hardware circuits.
